(19) United States
(12) Patent Application Publication
(10) Pub. No.: US 2016/0256279 A1
(43) Pub. Date: Sep. 8, 2016

(54) Patient-Specific Implant for Bone Defects and Methods for Designing and Fabricating Such Implants

(71) Applicants: Glenn Sanders, Sand Lake, NY (US); S. Alex Paolicelli, Pittsfield, MA (US)

(72) Inventors: Glenn Sanders, Sand Lake, NY (US); S. Alex Paolicelli, Pittsfield, MA (US)

(73) Assignee: Union College, Schenectady, NY (US)

(21) Appl. No.: 15/058,285

(22) Filed: Mar. 2, 2016

Related U.S. Application Data
(60) Provisional application No. 62/126,955, filed on Mar. 2, 2015.

(51) Int. Cl. A61F 2/28; G05B 19/4099; G06F 19/00

(52) U.S. Cl. CPC A61F 2/28; G06F 19/3437; G05B 19/4099; A61F 2002/30011

(57) ABSTRACT

The present disclosure relates to patient-specific bone implants and methods for designing and making such implants. A method includes obtaining an image of a bone having an injured, diseased, or degenerative portion; determining in the image the margins at each end of the injured portion of the bone; transforming the image into a three-dimensional model; conducting a virtual surgery to remove the injured portion of the bone and create a virtual bone gap in the image; designing a patient-specific implant to fit the virtual bone gap, wherein the designed implant includes a framework having a porosity sufficient to allow blood entry through the framework and having mechanical properties similar to that of bone; and fabricating an implant based on the designed implant. Optionally, bone regeneration material is placed within the framework.

### CROSS REFERENCE

This application claims the benefit of the filing date of U.S. Provisional Patent Application Ser. No. 62/126,955, filed Mar. 2, 2015, which is hereby incorporated by reference in its entirety.

### FIELD

The present disclosure relates to bone implants and methods for making such implants, and in particular to patient-specific bone implants and methods for making such.

### BACKGROUND

Certain orthopedic diseases and traumatic occurrences result in segmental bone defects, a condition in which a bone is left with a substantial gap. This gap is traditionally filled with bone graft, however there are limitations associated with this treatment. In particular, autografts can leave the patients with significant morbidity at the donor site, while allografts may result in the body's rejection of the foreign substance and/or may fail before becoming integrated with the host bone. As a result, porous scaffolds may be inserted into the void to promote bone growth.

The current gold standard to treat segmental bone defects is through the use of an allograft (cadaveric tissue). Negative outcomes can occur as the body may “reject” the foreign tissue. Once inserted, it is desirable for the native bone tissue to adhere to the allograft via bony ingrowth. This process can be slow in allografts and, in most cases, may never occur. As a result, the allograft is subjected to a large amount of mechanical loading during everyday activity and may not be able to withstand the lifetime of mechanical loading in the body before failure occurs.

An alternative to allograft is the use of a megaprosthesis. Insertion of a megaprosthesis involves the removal of a larger section of bone as well as the removal of a portion of the joint. For example, if bone cancer is present in the distal femur, the surgeon would remove the entire bone tumor including uninfected margins as well as the femoral portion of the knee joint. Replacement of the joint with a megaprosthesis is less than desirable, as these implants have a lifespan of approximately 10-15 years, and in many cases, these patients have a longer life expectancy. Also, the surgery associated with inserting these implants is highly invasive.

### SUMMARY

In accordance with one aspect of the present disclosure, there is provided a method for the design and fabrication of a patient-specific bone implant including obtaining an image of a bone having an injured, diseased, or degenerative portion; determining in the image a margin at each end of the injured portion of the bone; transforming the image into a three-dimensional model; conducting a virtual surgery to remove the injured portion of the bone at the margins and create a virtual bone gap in the image; designing a patient-specific implant to fit the virtual bone gap, wherein the designed implant includes a framework having a porosity sufficient to allow blood entry through the framework, for example, to contact bone regeneration material placed within the framework, and including mechanical properties similar to that of bone; and fabricating an implant based on the designed implant.

In accordance with another aspect of the present disclosure, there is provided a bone implant including an implant having a framework including porosity sufficient to allow blood entry through the framework, for example, to contact bone regeneration material placed within the framework, and having mechanical properties similar to that of bone.

These and other aspects of the present disclosure will become apparent upon a review of the following detailed description and the claims appended thereto.

### BRIEF DESCRIPTION OF THE DRAWINGS

FIG. 1 is a plan view of a femur with an implant in accordance with the present disclosure;

FIG. 2 is a perspective view of an inferior cap of an implant in accordance with the present disclosure;

FIG. 3 is a perspective view of a superior cap of an implant in accordance with the present disclosure; and

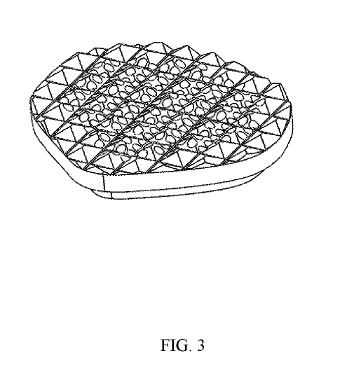

FIG. 4 is a perspective view of a V19.2 mm shell of an implant in accordance with the present disclosure.

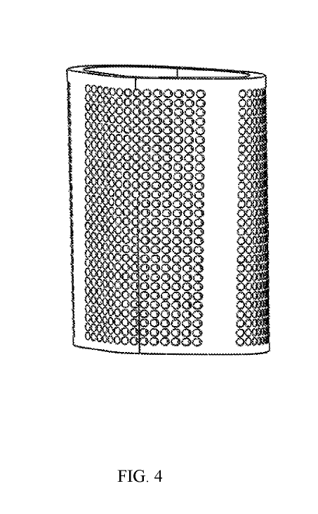

### DETAILED DESCRIPTION

The disclosure includes patient-specific bone implants and methods for designing and making such implants. The implant can be a framework of any design that has a strength and porosity to allow for bony ingrowth and the maintenance of bone nutrition. The porosity is designed such that the mechanical properties of the implant mimic that of bone and allow for bone regeneration. The overall porosity (independent of pore size) is above about 40%. In an embodiment, the porosity includes a value within the range of from about 40% to about 85%, preferably between about 70% and about 80%. The pores are designed to allow blood entry through the framework of the implant. For example, the pores allow blood entry through the framework enabling access to bone regeneration material placed within the implant. The pore shapes can have a cross-sectional geometry that is of any two-dimensional shape and size. Preferably, the pores run continuous through the implant framework. In an embodiment, the pores are circular having a diameter between about 0.5 to about 2 mm. In an embodiment having about 1 mm pore diameter, the density includes a value within a range of from about 30 to about 80 pores/cm². In an embodiment having about 0.5 mm pore diameter, the density includes a value within a range of from about 100 to about 180 pores/cm². In an embodiment having about 2 mm pore diameter, the density includes a value within a range of from about 9 to about 25 pores/cm². Pores may be aligned in any orientation and, in one embodiment, run in three orthogonal planes, e.g., aligned with the coronal, sagittal, and axial planes of the bone. In an embodiment, the pores are aligned with the axial and radial planes of the implant.

The mechanical properties of the implant mimic that of bone and include an effective elastic modulus of less than about 25 gigapascals (GPa). The effective elastic modulus is defined as the slope of the stress-strain curve of the implant as a whole. In this application, the cross-sectional area, which is used to derive stress, is defined as the global cross-section of the implant and does not incorporate the subtraction of the area of the whole. In an embodiment the effective elastic modulus is a value between about 1 and about 20 gigapascals. The mechanical properties of the implant mimic that of bone and include yield strength of less than about 800 megapascals.

In an embodiment, there is a large cavity in the volumetric center of the implant, which can accommodate the insertion of bone graft material during surgery. Bone graft material is defined as allograft, autograft, xenograft, or other synthetic or natural bone regeneration material. Graft may be inserted via injection through a pore or aperture in the implant. In an alternative embodiment, the implant is modular such that the implant has a superior end cap and inferior end cap. The end cap may be fabricated from the same material as the rest of the implant and may be fabricated by traditional methods or additive manufacturing methods. The end caps may or may not contain a plurality of pores.

In an embodiment, the cavity contains struts, and is substantially more porous than the shell of the implant. In an embodiment, there is no hollow region and the end caps are solid and non-removable. In an embodiment, the end caps of the implant, which are in contact with the adjacent bone, can be removed to insert osteoinductive, osteoconductive, or osteogenic material, such as bone graft or bone morphogenic proteins, and then replaced prior to or during surgical insertion. In an embodiment the bone graft material can be injected through the pores or an aperture of the implant. In an embodiment, the end caps have a roughened surface or contain ridges, to increase surface area in contact with adjacent bone and to improve fixation. The end caps may contain a shoulder that mates with the superior and/or inferior aspect of the implant. The end caps may also contain a portion that mates with the hollow region of the implant.

The implant is affixed to the adjacent bone by techniques generally known in the art. A traditional bone plate can be attached to the adjacent bone via bone screws to stabilize the region. This plate can also be attached directly to the porous implant via apertures designed to accommodate bone screws. Intramedullary rods can also be used to stabilize the region using traditional insertion methods. In this case, the end caps would be removed and the intramedullary rod would be inserted through the cavity of the implant. In an embodiment, the end caps would contain short rods that would be placed into the adjacent intramedullary canal during surgery. Surgical wire or cables may also be used to affix the implant to the adjacent bone and/or a bone plate, a bone screw, or a bone anchor.

The implant can be manufactured out of any suitable biocompatible material, including but not limited to titanium, silver, stainless steel, cobalt chrome, polyetheretherketone, or any combination thereof and using additive manufacturing (e.g., 3D Printing) techniques, such as direct metal laser sintering (DMLS) or Electron Beam Melting (EBM). In the preferred embodiment, the implant is manufactured out of titanium alloy (Ti-6Al-4V). The specific combination of the material selected and the pore design allows for the implant to behave mechanically like bone.

The implant is designed to fit a gap specific to an individual patient using the following methods. A computed tomography (CT) scan, MRI, or multiple x-rays is taken of the patient's injured bone. The surgeon or someone skilled in the art would then define where the diseased or traumatized bone is located and what bone would be resected during surgery. The scan and surgical information would then be brought into an imaging processing software, such as Materialise (Leuven, Belgium), where a threshold would be applied, based on the Hounsfield units of the present bone, so that the bony tissue is isolated from the soft tissue. This selection may then be brought into a computer aided drawing (CAD) software program, such as Solid Works (Waltham, Mass., USA), so that the injured, diseased, or degenerative bone could be manipulated and removed via a “virtual surgery.” A patient-specific implant would then be designed using standard CAD techniques to fit the void of the bone that was removed during the virtual surgery. Pores would then be added to the implant using standard CAD techniques. The computer model of the implant would then be uploaded to a machine that directly manufactures the implant via additive manufacturing methods. An alternative fabrication would be to use CAD to design the corresponding patient-specific mold of the implant so that the implant could be manufactured using traditional methods such as casting or injection molding. The implant would then be removed and go through standard post-processing such as sand blasting, surface coating, chemical etching, sterilization, etc. to prepare it for surgical implementation. The implant can also be coated with an osteoinductive material, such as hydroxyapatite or calcium phosphate, or can be filled with such materials or other graft materials prior to insertion.

Patient-specific implants, such as described above, can be used for any orthopedic disorder or disease where a segmental bone defect is present. In particular, bone cancer patients and high energy fractures (which often occur in the military) often result in the loss of a large segment of bone, which can accommodate such an implant. Spinal fusion surgeries also require a scaffold, or cage, to stabilize a region and allow for bone to bridge two adjacent bones.

The invention will be further illustrated with reference to the following specific example. It is understood that this example is given by way of illustration and is not meant to limit the disclosure or the claims to follow.

### EXAMPLE 1

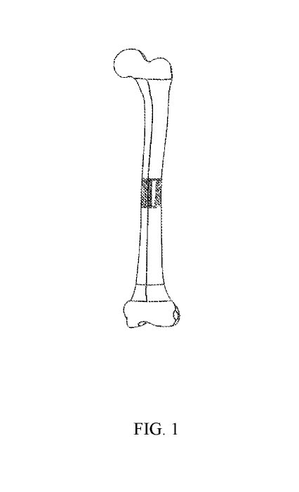

Methods: Radiograph and computational tomography (CT) data were received of an anonymous patient showing a lesion in the proximal one-third to mid diaphysis of the left femur (FIG. 1a). Using the radiograph and CT data, the tumor was located and acceptable margins for resection were identified. Using a software program Mimics, developed by Materialise (Leuven, Belgium), we transformed CT scan data of the patient's cancerous femur into a three-dimensional model. Using Mimics, we conducted a “virtual surgery,” at which point the tumor was resected. Using the CAD package 3-Matic, also developed by Materialise, a patient-specific titanium (Ti-6Al-4V) scaffold was designed to fill the bone gap. Pores were designed into the implant in the axial, coronal, and sagittal planes to promote osseointegration. Finite Element Analysis was used to assess the mechanical properties of the implant under two physiologic conditions: patient standing and gait. In both scenarios, a force vector was passed through the most superior portion of the femoral head and was in a trajectory with the lateral condyle of the distal femur (FIG. 1e). Two porosities and three pore sizes (1 mm, 1.5 mm, and 2 mm) were tested. A titanium (Ti-6Al-4V) implant was manufactured using the additive manufacturing method, Direct Metal Laser Sintering (DMLS). The testing conditions of the physical implant were then replicated and the strains of the implant were measured using Photogrammetry to validate the Finite Element Analysis.

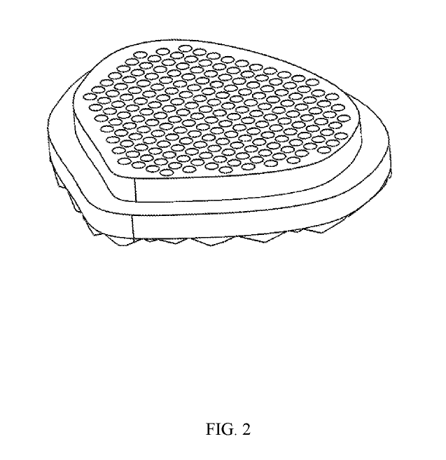

In all studies, titanium alloy (Ti-6Al-4V) properties were applied to the implant, the distal portion of the implant was fixed in plane. Peak stresses and the effective elastic moduli were measured for three different pore shapes (circular, square, and triangular) of equal cross-sectional area and pore quantity, as well as three different pore orientations: axial only, transverse only, and combined axial and transverse in 3 orthogonal directions. Overall stress distributions were also characterized. Pore density was increased until the modulus of the implant reached that of cortical bone (10-21 GPa). Experimental Validation: Based on the optimized computational design, an implant was fabricated using Direct Metal Laser Sintering (FIG. 2). Scanning electron microscopy (SEM) was used to characterize the pore diameters in the transverse plane. Due to size constraints, pores in the axial direction were measured using an optical microscope. Following geometric measurements, axial compression was applied (InstruMet Corporation, Union, N.J.) to the implant up to 600 N and 1200 N. Four data sets for each force magnitude were collected and the average effective elastic modulus was used to compare to computational models.

Results: Using the radiograph and CT data, the margins of the tumor were identified as 80 mm distal to the greater trochanter and 140 mm proximal to the lateral condyle of the femur. The virtual surgery left a 150 mm void in the patient's femur (FIG. 1b). We successfully were able to design a patient-specific implant to accurately fit that void (FIG. 1c). We then used the CT data to design a patient-specific fixation plate that was fixed to the implant and was attached to the proximal and distal portions of the femur (FIG. 1d). This plate was used to stabilize the bone-implant construct. Finite Element Analyses showed all stresses remaining below the endurance limit of Ti-6Al-4V, with the exception of the 1.5 mm pores during gait. All maximum stresses occurred locally at the bone-implant junction, while most of the stresses in the scaffold remained at 1-10% of that endurance limit.

Pore shape of equivalent cross-sectional area had minimal effect on the elastic modulus; however, stress concentrations around the edges of the square and triangle pores were 59% higher than the stress concentrations around the circular pores. Axially applied pores were more effective at lowering the elastic modulus than transversely applied pores; although, a combination of both patterns was most effective and was necessary to reduce the modulus of the titanium implant to that of bone. Based on these conclusions, an optimized femoral implant was designed with 1 mm circular pores applied both axially and transversely and a global porosity of 54%. The final computational design had an effective elastic modulus of 17.6 GPa, which is within the accepted values of cortical bone. SEM and Optical microscopy revealed that the average pore diameter of the DMLS implant were 0.84 mm. It was also observed that pores in the transverse plane were smaller than those in the axial plane (and also had a consistent deposit of material disrupting the circular shape The elastic modulus of the implant was measured to be 20.8 GPa,

Although various embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for the design and fabrication of a patient-specific bone implant comprising:
obtaining an image of a bone having an injured, diseased, or degenerative portion;
determining in the image a margin at each end of the injured, diseased, or degenerative portion of the bone;
transforming the image into a three-dimensional model;
conducting a virtual surgery to remove the injured portion of the bone at the margins and create a virtual bone gap in the image;
designing a patient-specific implant to fit the virtual bone gap, wherein the designed implant comprises a framework comprising a porosity sufficient to allow blood entry through the framework and comprising mechanical properties similar to that of bone; and
fabricating an implant corresponding to the designed implant.

2. The method of claim 1, further comprising placing bone regeneration material within the framework in contact with the porosity.

3. The method of claim 1, wherein the implant comprises a hollow portion adapted to accommodate bone regeneration material and removable end caps designed to be removable to accommodate the bone regeneration material.

4. The method of claim 1, wherein the mechanical properties comprise an effective elastic modulus of less than about 25 gigapascals.

5. The method of claim 1, wherein the mechanical properties comprise a yield strength of less than about 800 megapascals.

6. The method of claim 1, wherein the porosity comprises above about 40%.

7. The method of claim 1, wherein the framework comprises pores in three orthogonal planes.

8. The method of claim 1, wherein the framework comprises pores in axial and radial planes.

9. The method of claim 1, wherein the injured portion is a cancerous portion.

10. A bone implant comprising:
an implant comprising a framework comprising a porosity sufficient to allow blood entry through the framework and comprising mechanical properties similar to that of bone.

11. The product of claim 10, wherein the mechanical properties comprise an effective elastic modulus of less than about 25 gigapascals.

12. The product of claim 10, wherein the mechanical properties comprise a yield strength of less than about 800 megapascals.

13. The product of claim 10, wherein the porosity comprises above about 40%.

14. The product of claim 10, wherein the framework comprises pores in three orthogonal planes.

15. The product of claim 10, wherein the framework comprises pores in axial and radial planes.

16. The product of claim 10, wherein the implant comprises a hollow portion adapted to accommodate bone regeneration material and removable end caps designed to be removable to accommodate the bone regeneration material.
